(19) United States
(12) Patent Application Publication
(10) Pub. No.: US 2004/0087830 A1
(43) Pub. Date: May 6, 2004

(54) Arterial ventricular assist device

(76) Inventor: Jon Mark Strimling, Bedford, NH (US)

Correspondence Address: Jon Mark Strimling, 19 Mountain Road, Bedford, NH, 03110, US

(21) Appl. No.: 10/287,740

(22) Filed: Nov. 5, 2002

(51) Int. Cl. A61N 1/362

(52) U.S. Cl. 600/16

(57) ABSTRACT

A Ventricular Assist Device for human use is placed into the aorta or pulmonary artery to pump blood in series with the human heart rather than in parallel with the heart. The device may be used as a left ventricular assist device with a pump positioned in the aorta. The device also may be used as a right ventricular assist device with a pump positioned in the pulmonary artery or as a Bi-ventricular assist device with one pump positioned in the aorta and with another pump situated in the pulmonary artery.

### FIELD OF THE INVENTION

This invention relates to a pump placed in an artery in series with a ventile and useful as an arterial ventricular assist device (AVAD).

### BACKGROUND OF THE INVENTION

Currently available ventricular assist devices (VADs) are operative to assist the heart by drawing blood from either the ventricle of a heart or from an atrium and pumping the drawn blood into the aorta or into the pulmonary artery. With currently available VAD's some fraction of the overall circulatory blood flow passes through the VAD, while the remainder passes through the normal circulatory flow path, from the atrium through the ventricle to the artery. Therefore, currently available ventricular assist devices can be said to operate in parallel with the pumping action of the heart itself. To operate in this manner, the assist device has to be connected between the atrium or ventricle of a heart and the aorta or pulmonary artery. The device is so positioned by attaching the device between a single opening in each of the atrium or ventricle and in the aorta or pulmonary artery.

One problem with this approach is the high arterial pressure into which the existing heart must continue to pump, particularly for the left ventricle pumping into the aorta. Since the strain on the heart is proportional to the arterial pressure, the reduction of that pressure is desirable.

### BRIEF DESCRIPTION OF THE INVENTION

In accordance with the principles of this invention, a centrifugal or axial flow pump is inserted into the flow path of an artery as an arterial ventricular assist device. The device works in series with the existing heart, reducing both the arterial pressure and the strain on the heart's myocardium. This is in contrast to the prior art devices, which operate in parallel with an existing heart.

The device is monitored to synchronize the operation thereof with that of the heart.

### BRIEF DESCRIPTION OF THE DRAWINGS

FIG. 1 is a schematic representation of a prior art ventricular assist device (VAD);

FIGS. 2 and 3 are schematic representations of alternative embodiments in accordance with the principles of this invention;

FIG. 4 is a flow diagram of the operation of an AVAD in accordance with the principles of this invention; and

FIG. 5 is a representation of the human heart indicating the preferred location of the AVAD (and possible compliance chamber) in accordance with the principles of this invention.

### DETAILED DESCRIPTION OF ILLUSTRATIVE EMBODIMENTS OF THIS INVENTION

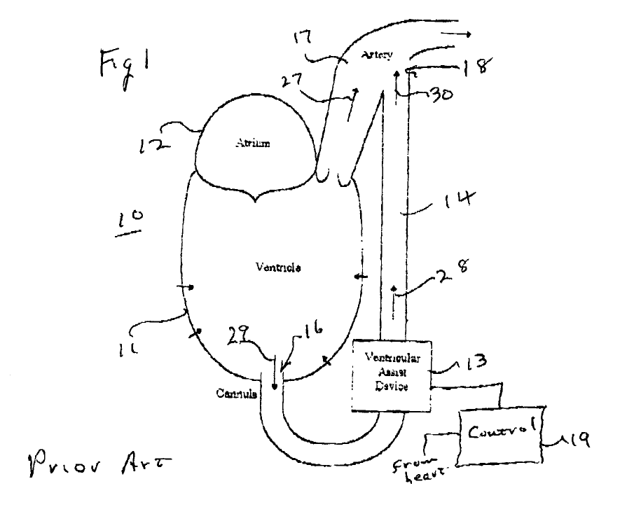

FIG. 1 shows a portion of a human heart 10 including a ventricle 11 and an atrium 12. In prior art arrangements, a ventricular assist device (VAD) 13 is connected into a cannula, which is attached at one end to the ventricle at 16 and at the other end to the aorta 17 at 18. The operation of the device is that of a mechanical pump which may be synchronized to the pumping action of the heart by a control 19.

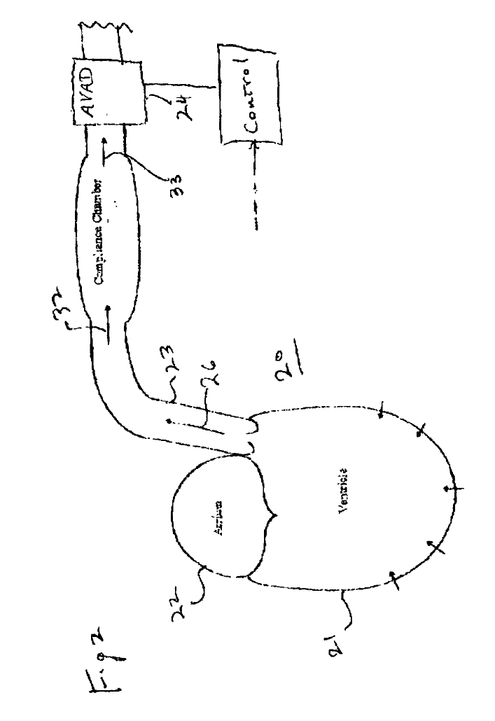

FIG. 2 shows a portion of a human heart 20 with a ventricle 21 and an atrium 22. Artery 23 is shown with the AVAD 24 positioned within it. Arrow 26 indicates the direction of blood flow from the ventricle and through the AVAD. It is clear that the device pumps blood in series with the action of the heart.

In FIG. 1 arrows 27, 28, 29 and 30 show that the prior art VADs pump blood in parallel with the pumping action of the heart. In FIG. 2 arrows 26, 32 and 33 show that, in accordance with the principles of this invention, with an AVAD blood flows in series with the pumping action of the heart.

The advantages of positioning a heart assist device in an aorta, in a pulmonary artery or in both is made clear by a comparison with a specific prior art device: The HeartMate™ left ventricular assist device made by Thermo-Cardio systems requires major surgery for installation of the device. Specifically, an incision from the neck to the abdomen for implantation, cutting open the apex of the heart, long cannulae that penetrate and traverse the diaphragm, large size, heavy weight, many moving parts, low efficiency, the potential for catastrophic failure, and an implanted compliance chamber that requires frequent servicing. Implantation is very complex and difficult.

The present invention requires a small incision in the chest only, for implantation, no surgery on the heart, very short cannulae and no penetration of the diaphragm. It is small size, lightweight, has only one moving part, high efficiency and a relatively safe failure mode in which blood does not stagnate. This invention employs a continuous flow type pump, which is installed either in the aorta or the pulmonary artery, or uses two or more pumps, one installed in each of these arteries or in their tributaries. The implantation procedure is simpler than that for the HeartMate™. The artery is cut, a section of the artery may be removed to make room for the pump and the pump is installed to reconnect the openings with such an orientation that the pump will move blood from the heart to the systemic circulatory system or to the pulmonic circulatory system or with two or more pumps, to both.

Axial and centrifugal pumps are available which can pump as much blood as the HeartMate™, and are much smaller, lighter, of simpler construction and more efficient design than the HeartMate™. This invention solves the problems of unnecessarily large size, heavy weight, multiple moving parts, large power consumption, the requirement for major surgery from neck to abdomen for implantation, the requirement for surgery on the heart, long cannulae, and the requirement for cannulae penetrating the diaphragm, by substituting a smaller, simpler, more efficient device. Its advantages include smaller size, lighter weight, only one moving part, lower power consumption, a relatively small incision in the chest for implantation, no surgery on the heart, short cannulae, no penetration of the diaphragm and no compliance chamber. In addition, in the event of catastrophic failure of the HeartMate™, blood can stagnate in the cannula and in the pump. In the event of catastrophic failure of an axial or a centrifugal pump, so long as the biological heart continues to pump there is no stagnation.

On embodiment of this invention employs an axial pump. Another embodiment employs a centrifugal pump. An axial pump has the advantage of small size. A centrifugal pump has the advantage of operating at lower speeds, which is less damaging to red blood cells in the bloodstream.

The output created by a ventricle is essentially given by the following equation:

Cardiac Output=(Stroke Volume)*(Heart Rate)

The parallel flow that is pumped through a VAD supplements the flow of the blood pumped by the ventricle. Most pulsative VADs are synchronized to beat at the same rate as the heart effectively increasing the stroke volume, thereby enhancing cardiac output. However, the arterial pressure and venous return pressures are fixed by the requirements of and resistance within the circulatory system. The arterial pressure is a strong determinant of the strain placed on the wall of the ventricle, according to the following simplified Laplace relationship:

Wall Tension=(Intraventricular Pressure)×(Radius)÷(Wall Thickness)

For a given heart with fixed dimensions, the maximum strain in the wall is directly proportional to the peak intraventricular pressure, which is in turn directly related to the arterial pressure. While existing VADs succeed in enhancing stroke volume, they do not decrease arterial pressure, and therefore do not decrease the tension in the wall of the heart.

An Arterial Ventricular Assist Device (AVAD) works on a different principle, whereby the full volume of circulated blood passes through the ventricle, but the pressure increase across the AVAD allows the ventricle to discharge at lower pressures while preserving the required systemic or pulmonic pressure downstream of the AVAD. An advantage is that the lower pressure results in lower strain in the wall of the heart than can be achieved with existing VAD's.

It is critical to synchronize the action of the AVAD with the pulsation of the ventricle. In a preferred embodiment, the centrifugal pump is accelerated so that as the ventricle contracts and discharges blood into the artery, the speed of the pump is at a maximum. When the semilunar valve closes, however, it is critical that the pump be slowed so as not to create a condition of suction in the artery upstream of the AVAD. While the pump is slowed, it is important not to entirely stop the AVAD because this could lead to blood stagnation and the risk of thrombosis.

The pulsation of the heart can be detected by monitoring fluctuations in electrical signals at the heart, pressure in the artery, or even by monitoring the power requirements of the AVAD itself. In a preferred embodiment, the circuitry of the centrifugal pump is such that power consumption and RPM are instantaneously fed back to an electronic controller. At a given rotational speed (RPM), the power required to turn the pump is a function of the flow rate of a fluid through the pump and the difference between the upstream and downstream pressures. By monitoring these parameters, the timing of the cardiac cycle can be determined, and this knowledge can be used to adjust the motor's drive circuitry.

In a control system, in accordance with the principles of this invention, the current voltage and rotational speed of the pump are monitored and controlled to enhance blood flow without ever creating a condition of suction. At a given rotational speed of the pump, the power supplied to the pumps' motor is related to the flow rate and to the pressure difference between the inlet and outlet of the pump. Using these parameters, the electronic control system monitors the pressure difference across the pump so as to maintain the appropriate systemic or pulmonic circulatory pressure while decreasing the upstream arterial pressure sufficiently to decrease the load on the heart without creating a condition of suction upstream of the device.

In the event that the electronic control system detects a condition of suction, it immediately adjusts the power to the motor, conceivably even reversing the voltage for an instant, to restore a positive inlet gage pressure. In this way a continuous flow type pump functions as a pulsative pump synchronized with the pumping of the human heart while avoiding the danger of collapsing an artery or collapsing the heart by creating suction.

A compliance chamber may be helpful in minimizing pressure extremes in the flow of blood through the AVAD without creating suction in the aorta or pulmonary artery. The compliance chamber is allowed to expand and contract in response to the pressures within the artery. In a preferred embodiment, the compliance chamber consists of a diaphragm sewn into one side of the artery that allows for expansion and contraction of the volume of blood contained in the artery between the heart and the AVAD. An alternative would be to use a balloon as a compliance chamber, inserted in the aorta or pulmonary artery between the heart and the AVAD. The presence of the compliance chamber allows the AVAD to continue to pump during diastole without creating a condition of suction in the artery. One or more independent pressure transducers placed in the artery upstream and/or downstream of the AVAD and/or in the compliance chamber may be helpful in providing pressure measurements for the control of the device.

The preferred embodiment of the AVAD is a centrifugal pump, but axial flow pumps or pulsative pumps may be reasonable alternatives. The principal advantages of centrifugal pumps with respect to alternatives are high reliability, low hemolysis and small size, which facilitates insertion into the body.

A centrifugal pump's high reliability comes from the small number of parts and the lack of reciprocating parts which are prone to fatigue. In a preferred embodiment, the centrifugal pump is supported on magnetic bearings. This minimizes the number of moving parts in contact with the blood plasma.

In accordance with this invention, a pump is inserted into a blood path so that the pump acts in series with the blood normally flowing in the blood path. Insertion into the aorta or pulmonary artery, as in FIG. 2, is only one embodiment. One end of the pump may be inserted within a ventricle of a heart so long as the action of the pump relieves the load on the heart muscle, and the flow of blood is in series with the ventricle.

Also, in accordance with this invention, the pump is modulated in sync with the existing blood flow. Consequently, when the heart muscle contracts and increases the pressure on the blood in an existing blood path, the pump senses the increase in pressure and is programmed to increase the power input driving the pump motor thus increasing the blood flow while reducing the load on the heart muscle.

The rotational speed of the pump may vary considerably in order to achieve the desired pressure response in the blood flow system. During periods of rapid deceleration of the pump, it may be necessary to use the motor circuitry to brake rather than drive the pump. In this scenario, regenerative braking may be advantageous. During periods where the pump needs to be slowed, the flow of blood through the pump is used to drive the pump, with the pump's motor windings acting as a generator. The energy generated in the pump's motor is then stored in a battery or capacitance circuit so as to be used during the next acceleration cycle. In a preferred embodiment, the pump is driven as a DC brushless motor. This has the advantages of high reliability and long life when compared to other motor technologies.

Thus, a pump positioned and operated in accordance with the principles of this invention takes over a major portion of the workload of the heart.

In an actual implantation in a human patient it might be difficult to cut the ascending aorta and insert an axial pump near the junction with the heart. If that turns out to be impractical, a pump can be inserted downstream in one of the branch arteries with perhaps a second pump in another branch artery. Both pumps could be controlled to operate synchronously or separately to assure that neither pump caused a dangerous drop in inlet pressure. And of course a third or more additional pumps could be inserted in other branch arteries if called for by the circumstances. In any case a separate pump or two or more pumps could be inserted in the pulmonary artery circuit also. Thus, the invention can be used as a ventricular heart assist device or as a bi-ventricular assist device.

The illustrative control system is based on information about the current and voltage of each pump motor, and not on information received from any separate pressure-monitoring device. But a separate pressure-monitoring device could theoretically be inserted in the blood path just upstream of each pump and connected to the control circuit so as to control the pump at optimum speed while reducing the danger of causing a vacuum and collapse of the inlet blood vessel or blood chamber.

It is to be understood that a pump herein can be used with or without an accessory compliance chamber. It is believed that a compliance chamber is desirable where it will fit comfortably in the body. However, it is not essential to the invention and may turn out to be superfluous.

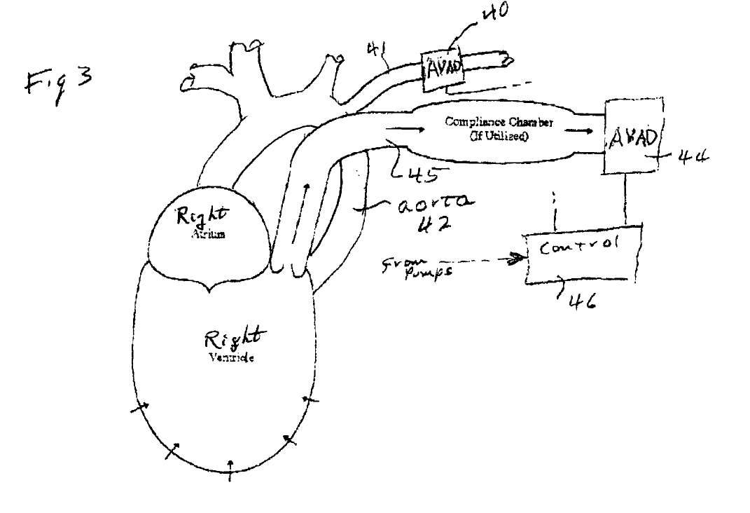

FIG. 3 illustrates the placement of a biventrical heart assist device consisting of AVAD 40 and AVAD 44. AVAD 40 is located in an artery 41 exiting an aorta 42 of a heart. AVAD 40 may be used alone or along with a AVAD 44 in the pulmonary artery 45. Control 46 operates, for example, responsive to information from pump(s) 40 (and/or 44) to operate the pumps synchronously or separately to avoid any drop in inlet pressure below a threshold that might be hazardous.

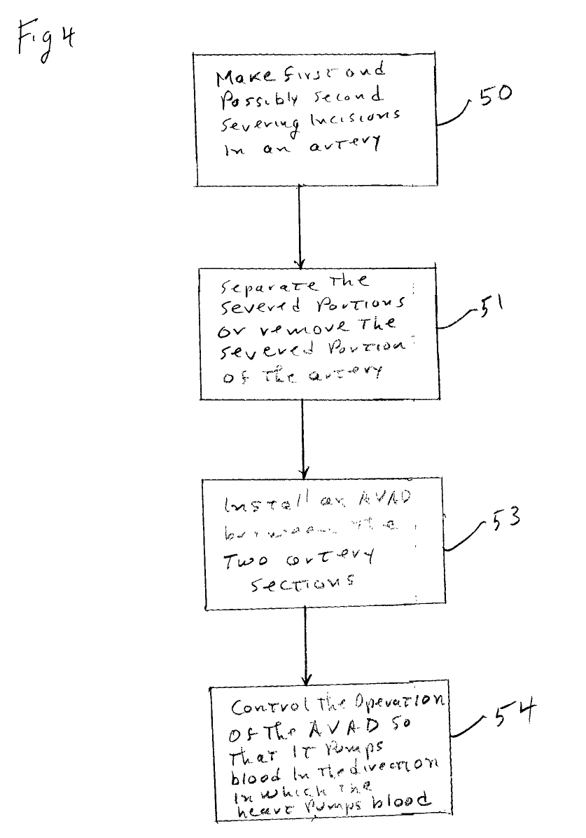

FIG. 4 is a flow diagram of the installation of an AVAD in accordance with the principles of this invention: Specifically, block 50 indicates the defining of a section of an artery by making first and possibly second spaced apart severing incisions in an artery. Block 51 indicates separation of severed sections or the removal of the section so defined. Block 53 indicates the placement of an AVAD in the artery between the severed sections or in place of the removed section. Block 54 indicates the operation of the AVAD in a manner to move blood in a direction in which blood normally moves in the artery.

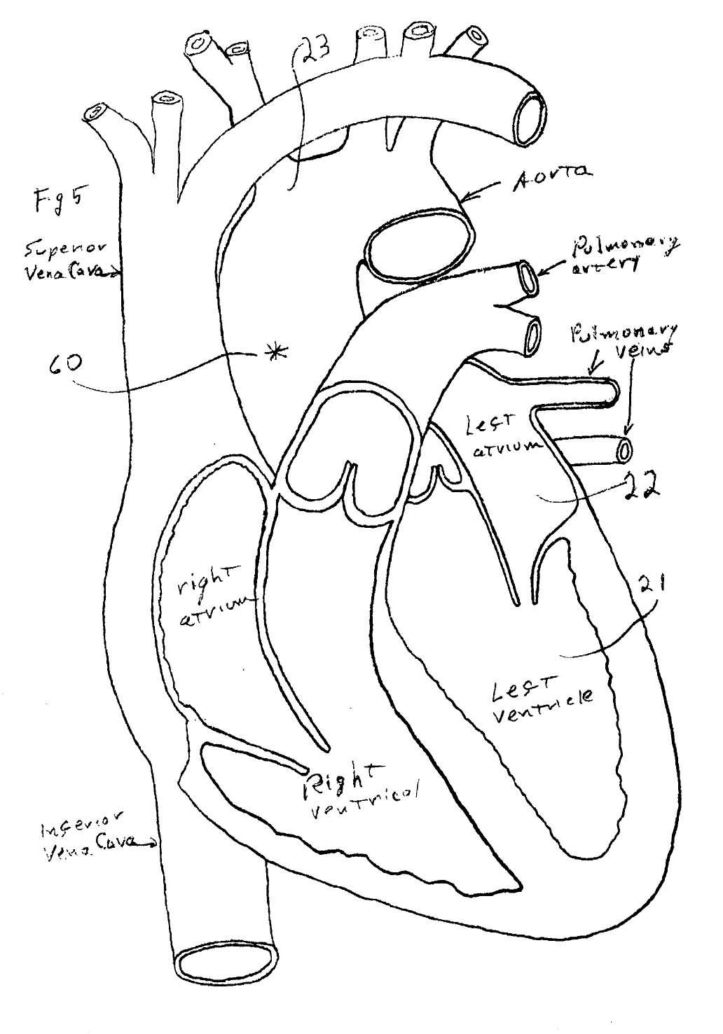

FIG. 5 is an anatomically correct diagram of the human heart showing the preferred location at asterisk 60 of an AVAD used as a left ventricular assist device. The left ventricle, left atrium, and the aorta in the figure are designated 21, 22, and 23 respectively as in FIG. 2 for facilitating a comparison between FIGS. 2 and 5.

While we have illustrated and described the preferred embodiments of our invention, it is to be understood that we do not limit ourselves to the precise constructions herein disclosed, and the right is reserved to all changes and modifications coming within the scope of the invention as defined in the appended claims.

What is claimed is

1. A system for assisting the operation of a functioning heart having a ventricle, said heart functioning to move blood along blood paths from said ventricle, said system comprising a first pump located within said blood paths and working in series with the pumping action of the heart.

2. A system as in claim 1 wherein said pump comprises an axial pump.

3. A system as in claim 1 wherein said pump comprises a centrifugal pump.

4. A system as in claim 1 wherein said pump is located in an aorta connected to said heart.

5. A system as in claim 1 wherein said pump is located in a pulmonary artery connected to said heart.

6. A system as in claim 1 wherein said heart is a human heart.

7. A system as in claim 1 also including a control system for synchronizing the operation of said pump with the pumping action of said heart.

8. A system as in claim 7 also including a control system that senses and monitors the current and voltage to the pump's motor in order to prevent a condition of suction up-stream of the pump.

9. A system as in claim 1 also including a compliance chamber located between said heart and said first pump.

10. A System as in claim 1 also including a second pump wherein said first pump is located in an aorta connected to said heart and said second pump is located in a pulmonary artery connected to said heart.

11. A system as in claim 10 including control means for synchronizing the operation of said first and second pumps with that of said heart.

12. A method for installing an arterial ventricular assist device in a body having an artery and a heart with a ventricle, said method including the steps of making at least a first incision in said artery for separating said artery into two sections, installing a pump between said two sections, and operating said pump such that the pump moves blood from the heart to the body's circulation system, in series with the normal flow of blood in said artery.

13. A method as in claim 12 wherein said artery is the pulmonary artery and the pump is inserted in said pulmonary artery.

14. A method as in claim 12 wherein said body includes an aorta and the pump is inserted in said aorta.

15. A method as in claim 12 including the step of operating said pump in a manner to avoid the presence of a vacuum in said artery.

16. A method as in claim 12 wherein said artery branches off said aorta and the pump is inserted in said artery.

17. A method as in claim 12 wherein the pump is inserted in an artery of the body branching off from said pulmonary artery.

18. A system as in claim 1 where the pump's motor is a DC brushless motor.

19. A system as in claim 1 where the energy used to brake the pump during the deceleration portion of its cycle is stored through regenerative braking by the pump's motor.

20. A system as in claim 19 also including a battery where the energy generated through regenerative braking is stored in said battery.

21. A system as in claim 19 also including a capacity where the energy generated through regenerative braking is stored in said capacitor.
